(19) United States
(12) Patent Application Publication
(10) Pub. No.: US 2011/0028759 A1
(43) Pub. Date: Feb. 3, 2011

(54) Process for the Purification of Organic Acids

(75) Inventors: Ooi Lin Lum, Singapore (SG); Govindharaju Venkidachalam, Singapore (SG); Yew Chin Neo, Singapore (SG)

Correspondence Address: WELLS ST. JOHN P.S., 601 W. FIRST AVENUE, SUITE 1300, SPOKANE, WA, 99201, US

(73) Assignee: HYFLUX IP RESOURCES PTE LTD, Singapore (SG)

(21) Appl. No.: 12/792,671

(22) Filed: Jun. 2, 2010

(30) Foreign Application Priority Data
Jul. 28, 2009 (CN) 200910152084X

(51) Int. Cl. C07C 51/48

(52) U.S. Cl. 562/513

(57) ABSTRACT

A process for recovery and purification of an organic acid from a fermentation broth containing a salt form of the organic acid, comprises the steps of subjecting the fermentation broth to one of ultrafiltration and microfiltration to form a first permeate, concentrating the first permeate to form a concentrated broth, subjecting the concentrated broth to a supported liquid membrane for extraction of lactic acid into a separate stream comprising an extracted solution, subjecting the extracted solution to activated carbon for colour removal, a cation exchange resin for demineralization, and an anion exchange resin for removal of anionic impurities to form a post polished organic acid, filtering the post polished organic acid to remove impurities above a predetermined threshold and concentrating the post polished organic acid to a desired concentration.

### FIELD OF THE INVENTION

The invention relates to a process for the recovery and purification methods to produce organic acids with higher heat stability, in particular, it relates to a process for the recovery and methods of purification of lactic acid with higher heat stability from fermentation broth containing lactic acid, with membrane technology.

### BACKGROUND OF THE INVENTION

The demand for organic acids, such as lactic acid, citric acid, ascorbic acid, gluconic acid, fumaric acid, etc., has been increasing over the years, owing to their extensive use in food, pharmaceutical, detergent or biodegradable plastic industries. Fermentation processes achieve production of organic acids on an industrial scale. Depending on the pH requirement of the bacteria strain used, the organic acids produced from the fermentation process is largely in salt form. The recovery of the organic acids from fermentation broth is a challenge to separation specialists.

Traditional processes for recovery and purification of organic acids from fermentation broth generally involves one or more precipitation stages. For example, under one known process for lactic acid production, the fermentation broth is generally heated to 70° C. to kill the bacteria and then acidified with sulfuric acid to pH 1.8. The precipitated salt is removed by filtration and the resulting liquid is treated with activated charcoal to remove any colouring materials. The clarified liquid is then ion exchanged and concentrated to 80%. Smell and taste can be further improved by oxidative treatment, e.g., with hydrogen peroxide. The lactic acid obtained at this stage is usually of consumable quality but not suitable as pharmaceutical grade. For pharmaceutical grade lactic acid, several additional purification steps would be necessary. A significant disadvantage of the traditional known process is relatively the high loss of lactic acid.

Alternative downstream processing techniques have been researched for more environmental friendly downstream processing. For example, electrodialysis membrane technologies have been proposed for recovery and purification of lactic acid. However, known electrodialysis membrane technology requires high quality feed and there are relatively high operating costs associated with the high electric current necessary for fast organic acids transport and a bipolar membrane used in such processes.

Another known organic acid purification technique is reactive liquid-liquid extraction, where the organic acids are extracted into an organic phase with a suitable carrier. The organic acids are then back extracted into an aqueous phase. U.S. Pat. No. 6,472,559 to Baniel et al discloses the use of phase transfer extraction of lactic acid from aqueous phase to water insoluble amine rich organic phase under highly pressurized carbon dioxide environment. The lactic acid is back extract to aqueous phase after removal of carbon dioxide environment. The drawback of this technique is the use of large quantity of organic solvent. Also, further purification steps often need to be carried out to remove contaminants.

Separation by liquid membranes is another technique used for purification of organic acids. The liquid membranes have been made of several different materials: e.g., liquid emulsion membranes, hollow fiber supported liquid membranes, and flat sheet supported liquid membranes. Liquid membranes separate the organic acid through liquid-liquid partitioning of the source stream with an organic phase that contains an active carrier. The organic acid is being extracted into the organic phase and it is then being back extracted into aqueous phase through partitioning of the organic phase with the stripping solution. The separation mechanism of supported liquid membrane (SLM) is different from other membranes. Known membranes separate components by size, while SLM extracts the component of interest via chemical means based on a facilitated transport mechanism. The chemistry of SLM is basically liquid-liquid extraction. A significant advantage of SLM over liquid-liquid extraction is that it requires very minimum organic solvent. However, the adoption of SLM in real industrial application has been limited by the stability (useful life) of the SLM. This is due to the lost of solvent and/or carrier to the aqueous phase. Water that is being transported across the membrane layer plays an important role in destabilizing the membrane. It would be desirable to provide an enhanced process for purification of organic acids using SLM membranes.

### SUMMARY OF THE INVENTION

In accordance with a first aspect a process for recovery and purification of an organic acid from a fermentation broth containing a salt form of the organic acid, comprises the steps of subjecting the fermentation broth to one of ultrafiltration and microfiltration to form a first permeate, concentrating the first permeate to form a concentrated broth, subjecting the concentrated broth to a supported liquid membrane for extraction of lactic acid into a separate stream comprising an extracted solution, subjecting the extracted solution to activated carbon for colour removal, a cation exchange resin for demineralization, and an anion exchange resin for removal of anionic impurities to form a post polished organic acid, filtering the post polished organic acid to remove impurities above a predetermined threshold and concentrating the post polished organic acid to a desired concentration.

From the foregoing disclosure and the following more detailed description of various preferred embodiments it will be apparent to those skilled in the art that the present invention provides a significant advance in the technology of organic acid purification. Particularly significant in this regard is the potential the invention affords for providing a process for production of organic acids which are heat stable. Additional features and advantages of various preferred embodiments will be better understood in view of the detailed description provided below.

### BRIEF DESCRPTION OF THE DRAWINGS

FIG. 1 shows a schematic of a filtration process for a raw fermentation broth in accordance with one embodiment.

FIG. 2 shows a schematic of a main process fluid concentration stage.

FIG. 3 shows a schematic of a main supported liquid membrane stage.

FIG. 4 shows a schematic of a supporting process fluid concentration stage.

FIG. 5 shows a schematic of a supporting supported liquid membrane stage.

FIG. 6 shows a schematic of a supporting ultrafiltration stage.

FIG. 7 shows a schematic of a polishing stage.

FIG. 8 shows a schematic of a product evaporation stage with a nanofiltration polishing stage.

FIG. 9 shows a schematic of a water reclamation stage.

FIG. 10 shows a schematic of a flow design of a supported liquid membrane.

FIG. 11 shows a schematic of an extraction process of the supported liquid membrane.

FIG. 12 shows a schematic of a recycling step of the organic acid for higher recovery.

It should be understood that the appended drawings are not necessarily to scale, presenting a somewhat simplified representation of various preferred features illustrative of the basic principles of the invention. The specific design features of the process for purification of organic acids as disclosed here, including, for example, the specific dimensions of the apparatus used at various stages, will be determined in part by the particular intended application and use environment. Certain features of the illustrated embodiments have been enlarged or distorted relative to others to help provide clear understanding. In particular, thin features may be thickened, for example, for clarity of illustration. All references to direction and position, unless otherwise indicated, refer to the orientation illustrated in the drawings.

### DETAILED DESCRIPTION OF CERTAIN EMBODIMENTS

It will be apparent to those skilled in the art, that is, to those who have knowledge or experience in this area of technology, that many uses and design variations are possible for the process for purification of organic acids disclosed here. The following detailed discussion of various alternative and preferred features and embodiments will illustrate the general principles of the invention with reference to a process suitable for use in purification as lactic acid. Other embodiments suitable for other applications will be apparent to those skilled in the art given the benefit of this disclosure.

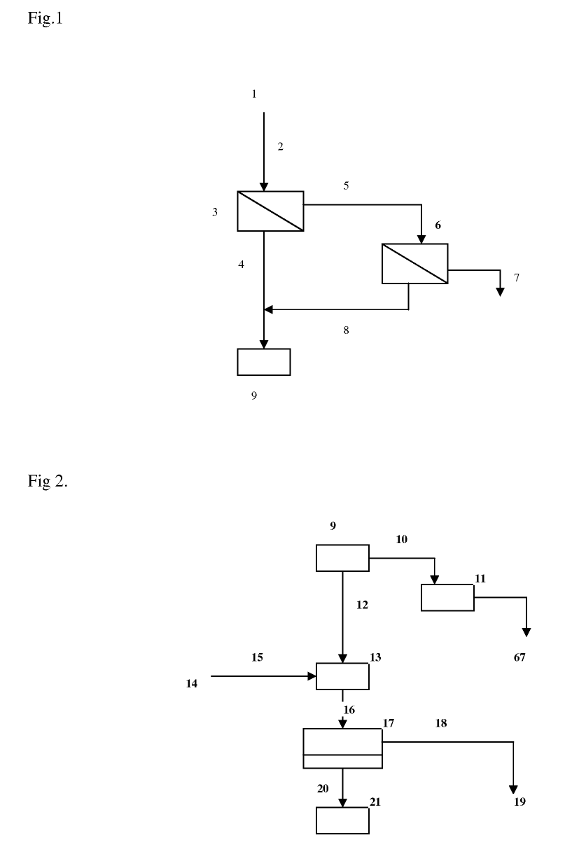

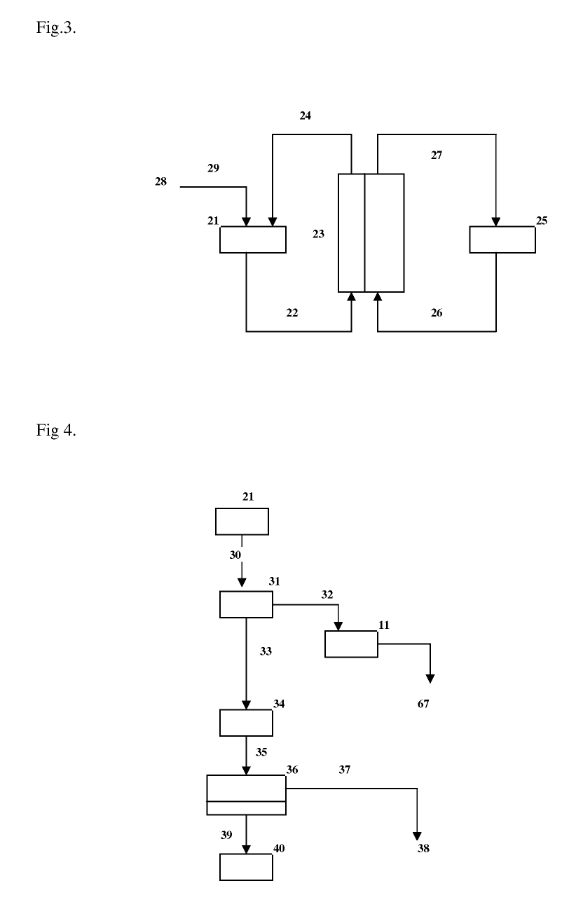

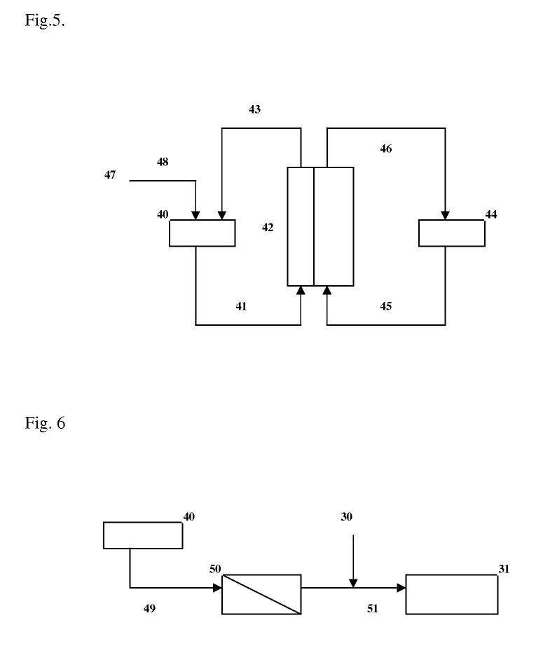

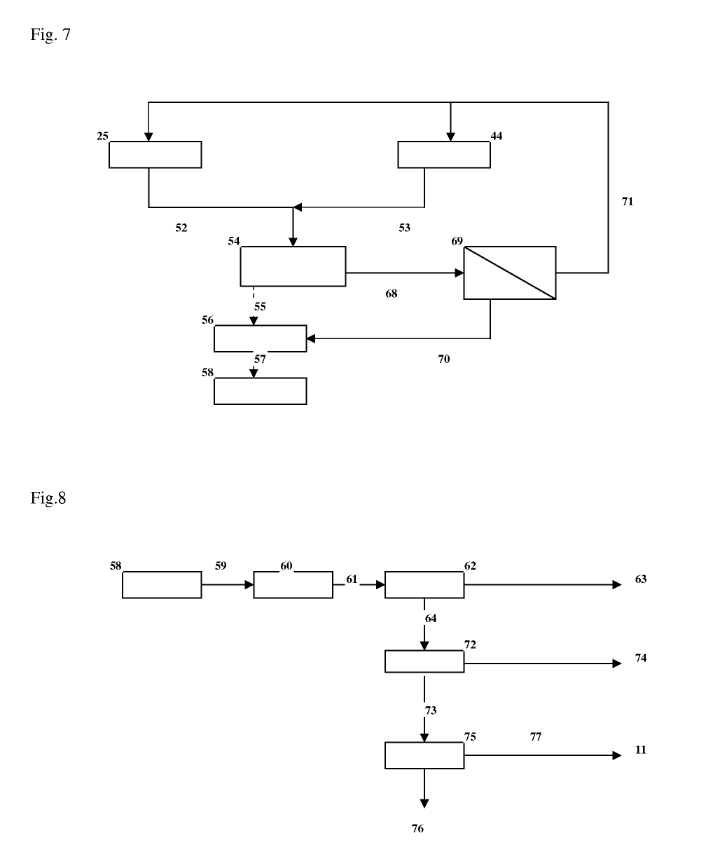

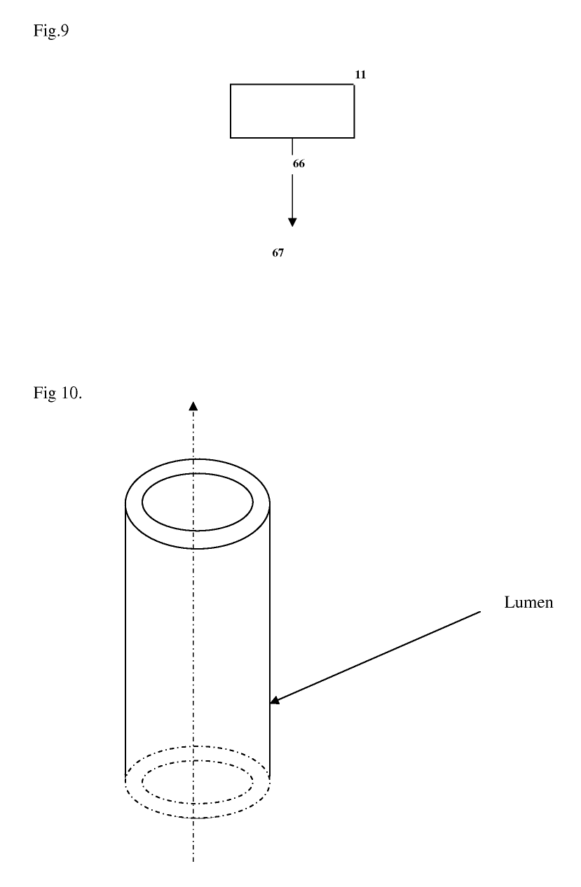

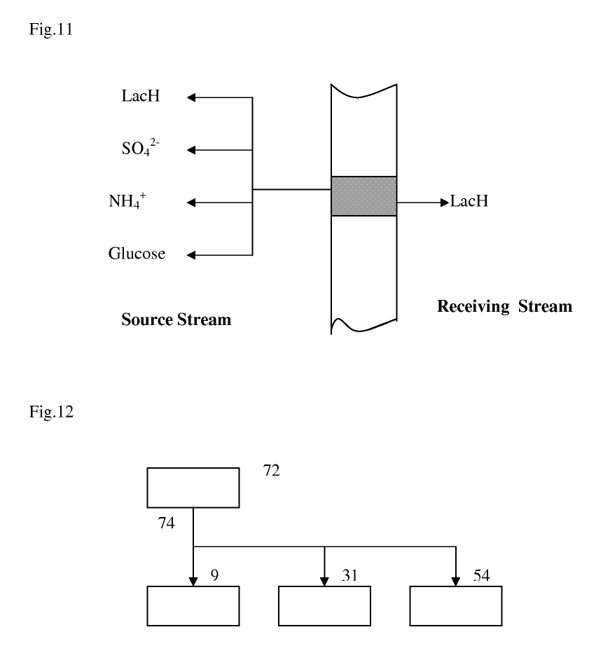

A process for recovery and purification of organic acids, in particular lactic acid, from a fermentation broth is disclosed. The process described herein can accept lactic acid fermentation broth with any concentration from 1% lactate or higher, in particular, 8% or higher. Turning now to the drawings, FIG. 1 shows a schematic of a process where a fermentation broth 1 is first fed into apparatus 3 via line 2. The fermentation broth may contain an organic acid such as lactic acid, and can be a salt form of the organic acid, such as a lactate. Apparatus 3 is preferably a membrane such as a microfiltration membrane 6 or ultrafiltration membrane 3, or both. The ultrafiltration membrane has filtration pores size of 0.1-0.01 μm. The ultrafiltration membrane can be in several configurations such as hollow fiber, tubular, flat sheet or spiral wound unit. In one form, a hollow fiber membrane is used which provides a good surface area to volume ratio. The ultrafiltration membrane of the present invention can be made of polymeric, ceramic or metallic materials. The ultrafiltration membrane acts as a form of barrier that blocks suspended solids, biomass, bacteria, etc. The filtration method to engage the ultrafiltration membrane can either be cross-flow or dead-end. In cross-flow filtration, a process stream flows parallel to the membrane. In dead-end filtration, process stream flow is perpendicular to the membrane. Only a portion of fermentation broth passes through the membrane with cross-flow filtration as compared to the dead end filtration method. The flow of fermentation broth parallel to the membrane is of sufficient velocity to wash the retained particulates away from the surface. Continual sweeping action minimizes the build up of particulates on the surface of the membrane, and advantageously extends operational life of the membrane. In the cross-flow method, the ultrafiltration membrane can achieve recovery rates of 30% to 99% of the desired organic acid, and typically 60 to 95% of the desired organic acid from the fermentation broth.

For higher recovery of the organic acid from the fermentation broth, a concentrated broth from apparatus 3 can be passed through line 5 into apparatus 6, the microfiltration membrane where remaining particulates and/or precipitates in the concentrate can be removed. The microfiltration membrane can have pores sizes ranging from 0.1 to 1 μm. The apparatus 6 can achieve about 50 to 90% recovery of the organic acids of the fermentation broth, which increases the overall recovery of organic acids (both UF and MF) to about 90 to 99% of the total in the fermentation broth. Further recovery of the lactic acid is achieved by microfiltration (MF) of lactic acid fermentation broth with addition of water into the feed. This process is called diafiltration. Combination of MF and diafiltration is used to enhance recovery of lactic acid. The microfiltration membrane can be in several configurations such as hollow fiber, tubular, flat sheet or spiral wound unit and can comprise polymeric, ceramic or metallic materials. Alternatively, in another embodiment, a combined MF-diafiltration process can be adopted to purify the fermentation broth directly to achieve more than 99% recovery of the lactic acid without subjecting the fermentation broth to ultrafiltration. The resultant of this first step is a first permeate.

The next step in the process is shown in FIG. 2. Here lactic acid can be recovered from the first permeate by concentration to form a concentrated broth to improve the recovery/extraction rate. An evaporator 9 is used to concentrate the first permeate to a concentrated broth. In particular, the organic acid content of the concentration broth may be 20-60%, more particularly 30-55%. The distillate obtained after evaporation process typically contains less than 0.5% of lactate. The distillate is practically water with some volatile organic carbons (VOCs) and traces of lactic acid and is easily clarified by passing through an activated carbon column (apparatus 11 as shown in FIG. 2) via line 10 to generate grade one quality water 67. Alternatively, the distillate can be reused in the fermentation broth.

In situation where the fermentation broth's initial pH is higher than its pKa (for example, lactic acid has a pKa=3.86), the concentrated broth requires an additional step of acidification. An acidification agent 14 (as shown in FIG. 2) suitable for use in the present invention is an inorganic acid such as hydrochloric acid or sulfuric acid. Sulfuric acid can be used as it does not carry much fume and moisture and thus would not cause much reduction in lactate concentration. The objective of acidification is to convert the salt of the organic acid in the fermentation broth to the organic acid. In general, pH is the controlling factor for the adjustment. The fermentation broth usually has pH of 5 to 6.5, and should be adjusted to lower than the pKa of the organic acid, in particular 1.5 to 3.8 for lactic acid broth, and yet more preferably 2 to 3.6. If the fermentation broth already reached a low pH, no further acidification is required to create an acidified broth. The amount of acidification agent 14 required is dependent on the initial pH of the fermentation broth. Upon cooling of the fermentation broth in tank 13, inorganic salt 19 may precipitate out of the solution. The inorganic salt 19 formed is dependent on the base and the acidification agent 14 used to control the fermentation pH during fermentation. For example, if ammonium hydroxide is used for controlling fermentation pH and sulfuric acid is used for the acidification, the inorganic salt 19 formed would be ammonium sulfate. Another reason that sulfuric acid is a desirable acidification agent is that sulfate salt generally precipitates easily as compared to other acidification agents. Equation 1 shows an example of the reaction occurring when sulfuric acid is introduced to a solution containing a lactic acid salt.

### Acidification of Ammonium Lactate to Lactic Acid with Sulfuric Acid

2LacNH₄+H₂SO₄→2 LacH+(NH₄)₂SO₄ Equation 1

The acidification process to create an acidic broth with the addition of an acid is exothermic and thus it generates heat and resulting in an increase of solution temperature. After the solution is cooled to at least approximately 50° C. and more preferably 25° C., a salt may precipitate out. In the example above, ammonium sulfate will start to precipitate out. A quantity of sulfate will crystallize out when the solution is cooled to room temperature (25° C.). In fermentation, lactate salt can be calcium lactate, sodium lactate or ammonium lactate. During acidification using sulfuric acid, corresponding sulfate salt will be produced. Any salt formed during this process is filtered off, optionally by a centrifuge. In general, salt forms a substantial quantity if (i) the initial fermentation broth has a pH of 5 or higher (in sodium or ammonium lactate); (ii) sulfuric acid is used as the acidification agent; and (iii) the concentration of the fermentation broth has been increased to more than 20% during the step of concentrating the first permeate. The separation step separating the concentrated broth from the salt is effected through apparatus 17. Apparatus 17 can be filter press or any other solid-liquid separators. Preferably the remainder of the steps of the process are carried out near ambient conditions.

The (filtered, acidified and) concentrated broth can be filtered as described above. The concentrated broth generally contains low levels of suspended solids. Depending on the concentration, where lactic acid is intended for recovery the concentrated broth can be a clear solution or dark viscous liquid more than 20% of lactic acid, in particularly 20-65% lactic acid concentration. The concentrated broth is delivered to a tank 21 shown in FIGS. 2-4. Recovery of lactic acid takes place when the filtered acidified broth (20) is fed into an apparatus 23 as shown in FIG. 3. The apparatus 23 used for the extraction of lactic acid from the acidified, concentrated broth is a supported liquid membrane (SLM).

The SLM 23 comprises an organic layer that consists of suitable components that are impregnated on another membrane (base membrane), such as ultrafiltration (UF) or microfiltration (MF) type membrane. In one form, MF is used due to its higher pore area density. The base membrane used in SLM has hydrophobic nature and can comprise a hydrophobic polymer, such as polypropylene (PP), polyvinyldifluoride (PVDF) and polyethylene (PE); amphoteric polymer such as polysulfone (PSF), polyether sulfone (PES) and polyvinyl sulfite (PVS). Generally a hydrophobic polymer is suitable for use as the base membrane; in the most preferred form, PP polymer is used, owing to its highly hydrophobic nature, relatively low cost, good mechanical properties and good chemical stability.

The SLM 23 has an organic layer impregnated into the base membrane which stabilizes the base membrane by containment of pores (here, micropores) of the membrane during rugged operations. The organic layer can contain four components: a carrier, a co-extractant, a diluent and a stabilizer. The carrier contains a water insoluble amine, in particular, a primary, secondary, tertiary aliphatic or aromatic amine. More preferably, it comprises an amine with at least one alkyl side chain of C₄ to C₂₄. In the most preferred form the carrier is a tertiary aliphatic amine with alkyl chains of C₈-C₁₂.

The co-extractant is a liquid that assists the carrier in the organic acid extraction process. The co-extractant can comprise an aliphatic alcohol that has little water miscibility, such as an alcohol with a carbon chain of C₂-C₂₉ and more particularly, it is an alcohol with carbon chain of C₆-C₁₀. The alcohol functionality can be at the end of the carbon chain (normal alcohol) or at a branch. The co-extractant can comprise, for example, either a linear alcohol with C₈-C₁₀ or a branched alcohol with C₆-C₉.

Diluents are added to the organic layer to dilute the concentration of the carrier so as to decrease the viscosity of the carrier to aid in the rate of extraction of the organic acid. In generally, any organic liquid that is compatible to the base membrane and not water miscible could be used. Suitable diluents include hydrocarbons, ketone, ether, or ester. Suitable hydrocarbons can comprise, for example, kerosene, methyl isobutyl ketone, mono-isobutyl ketone and butyl acetate may be used. Other suitable diluents will be readily apparent to those skilled in the art given the benefit of this disclosure.

The stabilizers help stabilize the organic components, i.e., extractants, co-extractants and diluents in the base membrane. The useful life of the SLM is dependent in part on the rate of organic component loss to the surroundings, i.e., to the aqueous phase. In known SLMs, this occurs within few hours. In accordance with a highly desirable feature, the stabilizer described herein has a non-ionic surface-active agent that has very little solubility in water and has a low aqueous surface tension. The stabilizer while in the organic composition acts as the barrel between the organic and the aqueous phase and therefore reduces the mixing of the two phases.

Three primary groups of stabilizers are suitable for use in the present invention: hydrocarbon based, silicone based and fluorocarbon based stabilizers. The non-ionic surface-active agent can be fluorocarbon based. The non-ionic surfactant is a form of surface-active agent without an ionic head group. The hydrophilic group of the fluorocarbon based surfactant is a non-ionic ethoxylated group and hence has low water solubility. The tail group of the fluorocarbon based surfactant is both hydrophobic and lipophilic. This ensures that the stabilizer will predominantly resident at solvent-aqueous interface. The boundary creates by the fluorocarbon based surfactant would limit the mixing of water with the organic solution in the membrane and thus reduce water transport across the membrane and prolong the stability of the SLM membrane. The non-ionic nature of the surface-active agent also acts as additional barrier to the ionic species and thus improves the selectivity of the membrane toward organic acid. Comparatively, the organic acid in its acid form would be less resisted by the surfactant, while inorganic acid such as sulfuric acid and hydrochloric acid are fully ionized in aqueous medium and thus would be restricted for entering the liquid membrane phase (since water transport is limited). This results in a highly desirable selectivity between organic acids and inorganic acid. In a typical experimental setup with liquid membrane composition of 0.01% stabilizer, the selectivity in favour of organic acids over inorganic acids can be as high as few thousands times.

Similarly, the restriction of water-liquid membrane interaction also reduces the transport of glucose across the membrane. The SLM of the present invention comprises a suitable selection of the extractants, co-extractants and diluents mentioned above can be stable as much as 180 days. In general, a stabilizer can be added in the range of 0.001-10%, with higher proportion of the stabilizer having more stable membrane but lower extraction rate. The most preferred stabilizer concentration is 0.005-0.020 ppm. The fluorocarbon based non-ionic surfactant has a general structure of R_{f}CH₂CH₂O(CH₂CH₂O)ₓH, where x is a number ranges from 0-25, and R_{f} is fluorocarbon group F(CF₂CF₂)_{y} where y is 1 to 20.

The carrier, co-extractant, diluents and stabilizers are mixed into a homogeneous phase before impregnated into the pores of the base membrane. The base membrane can be formed having a hollow fiber configuration. The apparatus 23 permits the flow of one stream along the lumen of the fibers while another stream along the shell side of the fibers. A more preferred arrangement is to let the source solution, i.e. the concentration broth, run along the shell side while the receiving solution (also referred to as stripping solution) along the lumen. Both solutions are re-circulating along the respective side: source solution along line 22 (as shown in FIG. 3) into apparatus 23 and along line 24 to bring the solution back to tank 21; the receiving solution transfer along line 26 into apparatus 23 and along line 27 to bring the solution back to the holding tank 25. The pH of the source phase is being maintained at lower than the pKa, such as 1.5 to 3.6 for lactic acid solution by acid 28 via dosing line 29. Acid 28 is generally the same as the acidification agent 14. The receiving solution could be water alone, or contains chemical such as hydrochloric acid or sodium carbonate. The most preferred receiving solution is plain water, as this reduces the polishing effect in later stages. The extraction processes involve:

(I) Protonation of Carrier with Organic Acid:

[R₃N]_{org}+[LacH]_{aq} [symbol] [R₃NH⁺Lac⁻]_{org}

During the protonation, the organic acid is being attached to the amine;

(II) Transfer of Lactic Acid Across the Organic Layer to the Receiving Solution Side.

The amine-lactic acid complex is transported across the organic layer from the source solution side to a receiving solution side. The transportation mechanism is either diffusion of the complex or hoping of the lactate molecules:

[R₃NH⁺Lac⁻]_{org}+[R₃N′] [symbol] [R₃N′H⁺Lac]_{org}+[R₃N]

where N′ is closer to the receiving end, and at the receiving end; and

(III) Deprotonation of Amine

[R₃N′H⁺Lac⁻]_{org} [symbol] [R₃N′]_{org}+[LacH]_{aq}

The lactic acid (or organic acid in general) is transferred from source solution to the receiving solution.

The ratio of the quantity of source to receiving solution is preferably from 1:1 to 8:1, and yet more preferably, from 1:1 to 4:1. The time of the extraction process depends in part upon the source to receiving ratio, organic acid concentration and the extraction apparatus (i.e. the supported liquid membrane). The extraction process should be stopped when source phase organic concentration is more than 20% higher than the receiving phase, since the extraction rate becomes undesirably slow. The receiving solution can be collected for further treatment. A fresh receiving phase is circulated in the system to further extract the lactic acid. After a few rounds of extraction, the source solution would contain less than 8% lactic acid, which would be less suitable for extraction as the extraction rate would become too slow. In one embodiment where the source to receiving ratio is 2:1, and source phase lactic acid concentration is 48% initially; the source solution lactate concentration would reduce to 7 to10% after six rounds of extractions of 3 to 5 hours each. The average lactic acid concentration in the receiving solution is 1 to 15%. Advantageously, the apparatus 23 has high selectivity for the organic acid. In general, the receiving solution has no significant amount of glucose, which is the raw material for the fermentation of lactic acid. The colour of the receiving phase is low relative to the source solution, since lactic acid is being extracted into a clean solution. Comparing with the clarified broth (after UF/MF), it could be 50 to 500 times reduction in colour. The high selectivity nature of the SLM ensures that the receiving phase contains low amounts of ionic impurities and practically independent of source phase ionic impurities concentration. In the execution of the preferred apparatus with initial source containing 48% lactate, pH 3.2, ammonium 4.0 to 4.5%, sulfate 10 to 20%, the receiving solution would contains ammonium 0.0001 to 0.05% and sulfate 0.0001 to 0.04%.

To improve the recovery, the concentrated solution can be sent to another evaporator 31 via line 30 for further concentration. The evaporation capacity of the apparatus 31 is roughly 5-8 times smaller than apparatus 9. The output from apparatus 31 can contains 15%-60% lactic acid, typically about 30-50%. As the solution already contains ammonium sulfate at near saturation point, ammonium sulfate might precipitate out during concentration, particularly when concentrated beyond 40%. The concentrated broth can be filtered in the similar manner as the previous process, via line 33 into a cooling tank 34 and out through line 35 into a filter press or any suitable solid liquid separation apparatus 36 to obtain a clear concentrated broth which is collected in tank 40 and ammonium sulfate crystal or any carbonized precipitate in 38. In this filtration step, no further pre-acidification is necessary as the concentrated broth is already at low pH. The clarified concentrated broth in tank 40 is then subjected to extraction of lactic acid with SLM of apparatus 42 using the same extraction method as described above for apparatus 23. The resulting solution can be discarded, or fed into apparatus 50 via line 49 for further ultrafiltration before directing the solution into apparatus 31 for further concentration.

All the receiving extracted solutions from the SLM processes (collected in tank 25 and 44) are combined to a stream that contains certain quantity of compound which adds colour to the extracted solution. The combined stream is fed into activated carbon column apparatus 54 (as shown in FIG. 7) where reduction of the colour of the solution takes place by the introduction of activated carbon to the extracted solution. Activated carbon binds to the colour containing compounds, removing them from the extracted solution.

As shown in FIG. 7, the decolourized extracted solution from apparatus 54 can be directed to apparatus 69 via line 68 for concentration. Apparatus 69 in general, can be any apparatus that can concentrate the organic acid by removing water from the broth solution, in particular, concentrating the feed solution from as low as 0.05% to a concentration as high as 50%, and in a more preferred embodiment, from feed concentration of 1-8% to output concentration of 8-10%. In the more preferred embodiment, apparatus 69, is a polymeric membrane which in its operating mode, only permits water of the extracted solution to flow through the membrane. The water permissibility of apparatus 69 can be pressure driven, vacuum driven and/or thermal driven. In the most preferred embodiment, a reverse osmosis (RO) membrane, which is a pressure driven membrane, is used in apparatus 69. The use of apparatus 69, concentrates the extracted solution to a higher concentration at much lower energy cost than a conventional evaporation apparatus. The pre-concentration by apparatus 69 advantageously reduces the volume that apparatus at subsequent steps need to handle. The water removed by apparatus 69 can be directed to apparatus 25 and 44 for use as the source of the fermentation broth or wherever else it is needed. Alternatively the step of concentration may be perform prior to the step of removal of colour.

The extracted solution, irrespective of whether subject to further concentration from apparatus 69, can then be directed to a series of columns for polishing to increase quality. The series of polishing column can consist of, for example i) a cation exchanger column for removal of cationic impurities (demineralization); ii) an anion exchanger column for removal of anionic impurities; iii) polishing colour removing resin or activated carbon column for removal of colour. These three columns can be operated in any sequence, although in the more preferred embodiment, cation exchanger column is preferably before anion exchanger column, while colour removing or activated carbon can be positioned before, after or in between cation and anion exchanger column.

The pre-concentrate broth from apparatus 69 or decolourized broth from apparatus 54 is directed to cation exchanger apparatus 56 via line 70 or line 55 respectively for removal of any trace of cationic impurities. In general, any strong cation exchange resin can be used in apparatus 56. Use of macroporous type cation exchange resin is one option. Besides removing the cationic impurities, the cation exchanger column 56 also further removes some or all of the colour of the broth. The demineralized lactic acid solution is then further treated with an anion exchanger apparatus 58 via line 57, where anionic impurities are removed. A weak anionic exchange resin is required in apparatus 58. In the one embodiment a macroporous type resin is used.

The steps of removal of colour may be repeated if needed. Also, the step of condensation may also be repeated after cation exchange, anion exchange and colour removal. For example, although the output extracted solution from the anion exchanger apparatus 58 generally contains no colour, in any process low level of colour persists after subjecting the extracted solution to the anion exchanger, the output from anion exchanger can be further subjected to colour removal in apparatus 60 that contains for example a polishing colour removing resin or activated carbon. The resultant extracted solution generally contains 7-12% of lactic acid depending on the initial concentration. If the resultant solution's concentration is lower than 10%, it is advantageous to subject the solution to another concentrating apparatus 62. Apparatus 62 can be any embodiment similar to apparatus 69. In one of the preferred embodiment, another RO membrane is used in apparatus 62. Apparatus 62 can concentrate the solution to 11-15% of lactic acid. The water 63 removed by apparatus 62 can contains from 0.1% to 6% of lactic acid depending on initial concentration and resultant concentration. The resultant extracted solution contains 11 to 15% organic acid. It can then be subjected to purification through apparatus 72. Apparatus 72 is a separation device that based on a molecular weight cut off. It removes larger molecular weight impurities above a predetermined threshold that might have pass through all previous processes. Apparatus 72 is preferably a nano-filtration membrane with molecular weight cut-off (MWCO) of between 100-300 Daltons, and more preferably with 100-150 MWCO for purification of lactic acid. Apparatus 72 allows lactic acid to pass through the membrane while retaining most of the impurities with molecular weight higher than its MWCO. It improves the colour value of the lactic acid solution when it is further concentrated to higher concentration beyond 75%.

The permeate from apparatus 72 can then be subjected to further concentration with a product evaporator, i.e., apparatus 75 as shown in FIG. 8. The concentration factor for apparatus 75 can be 20-40 times, more commonly 25-35 times. The concentrate 76 from the apparatus 75 can be directed to product packing sections. The concentrate 74 from the apparatus 72 may be subjected to either apparatus 9 or 31 for evaporation, or revert back to apparatus 54 to increase the recovery of the lactic acid as depicted in FIG. 12. This post polished organic acid may be treated with an oxidizing agent such as hydrogen peroxide to produce a heat stable organic acid—i.e., an acid resistant to discolourization at elevated temperatures.

FIG. 9 shows that the distillate from apparatus 14, 28 and 75 can be treated with apparatus 11 to remove the VOCs and trace quantity of lactic acid to generate grade-1 quality water 67. The quantity of 67 is generally sufficient to supplement 70-90% demand of the whole processes including washing of equipment. Alternatively, distillate from apparatus 14 and 28 could be use directly in preparation of fermentation broth, while the distillate from apparatus from 75 can be used as the receiving solution for the SLM.

The present invention is further illustrated by the following examples, which are not to be construed in any way as imposing limitations upon the scope thereof.

### EXAMPLE 1

### Ultrafiltration

253 L of fermentation broth was circulated in an ultrafiltration membrane system at feed pressure of 2 bars. The ultrafiltration membrane was polyether sulfone based hollow fiber membrane with an effective area of 3.5 m². The fermentation broth was fed and flowed in the lumen of the fiber. The reject pressure was controlled at 1.6 bar pressure. The trans membrane pressure was at 1.8 bars. The initial permeate flow rate was 1.9 L/min and declined to 0.5 L/min after 3 hours at 86% recovery. The average flux was 19.5 LMH. The suspended solid in the raw fermentation broth and the first permeate was 3.88 g/L and 0.005 g/L respectively. The concentrate of the first permeate had suspended solid of 49.78 g/L.

| Parameters | Unit | Feed | Permeate | Concentrate |
|---|---|---|---|---|
| Volume | L | 253 | 220 | 16 |
| Suspended solid | g/L | 3.88 | 0.005 | 49.78 |
| Lactate concentration | g/L | 108.9 | 107.2 | 99.7 |
| Turbidity | NTU | 4580 | 1.2 | 52000 |

### EXAMPLE 2

### Microfiltration

Sixteen liters of concentrated broth of ultrafiltration (UF) (i.e. microfiltration (MF) feed) was circulated in stainless steel MF membrane with titanium dioxide coating. The membrane had pore size of 0.1 μm. The MF feed had 49.78 g/L of suspended solid. The MF was operated at 3 bar trans-membrane pressure. The average flux was 80 LMH.

### EXAMPLE 3

### Concentration of First Permeate from 11% to 48%

100 L of first permeate was concentrated from 11% to 48%. The quantity of the concentrated broth recovered was 22.9 L, while 77.1 L was collected as distillate.

| Parameters | Unit | Feed | Concentrate | Distillate |
|---|---|---|---|---|
| Volume | L | 100 | 22.9 | 77.1 |
| Lactate concentration | g/L | 100-105 | 475-485 | <0.2 |

### EXAMPLE 4

### Acidification and Crystallization of Ammonium Sulfate

77.2 L of concentrated solution containing 48.6% lactate was acidified from pH 5.6 to 3.2 with 13.8 kg of sulfuric acid. 6.1 kg (wet weight) of ammonium sulfate crystal precipitated out after acidification and the solution was cooled to 25° C. After filtering off the ammonium sulfate crystal, 82.2 L of the acidified broth was recovered. Lactate recovery was up to 99.5%.

| | | Before | After acidify |
|---|---|---|---|
| | Unit | acidify | and filter |
| Volume | L | 77.2 | 82.2 |
| pH | | 5.6 | 3.2 |
| Lactate concentration | g/L | 486.8 | 454.7 |
| Sulfate concentration | g/L | 16.8 | 186.5 |
| Ammonium concentration | g/L | 79.7 | 61.5 |
| Lactate quantity | kg | 37.58 | 37.38 |

### EXAMPLE 5

### Extraction of Lactic Acid with Supported Liquid Membrane

The concentrated lactic acid solution with 40-48% lactate was extracted with hollow fiber supported liquid membrane (SLM) with 70 m² membrane area. The organic layer impregnated to the membrane contained 0.001-10% carrier, 99.9-90.0% co-extractant and diluents. Water was used as the receiving solution. The quantity of the receiving solution used was half the starting source solution volume per extraction that lasted 3 to 5 hours. The similar process was scaled up to industrial size module with effective membrane area of 300 m².

### EXAMPLE 6

### De-Colourization with Activated Carbon

A total of 77.2 L of extracted solution from SLM process was treated with an activated carbon column of 1 m length, 1.5″ column diameter and 0.8 kg carbon.

| | Unit | Before treatment | After treatment |
|---|---|---|---|
| Volume | L | 77.2 | 82.2 |
| Colour | Pt—Co | 2000-4000 | 300-500 |

### EXAMPLE 7

### Demineralization with Strong Cation Exchange Resin

A total of 82.2 L of extracted lactic acid solution that had been treated with activated carbon was treated with a macroporous strong cation exchange resin column of 1 m length, 1.5″ diameter and 0.7 kg resin.

| | Unit | Before treatment | After treatment |
|---|---|---|---|
| Volume | L | 82.2 | 84.29 |
| pH | | <1 | <1 |
| Lactate concentration | g/L | 116-121 | 113-118 |
| Sulfate concentration | g/L | <0.8 | <0.8 |
| Ammonium concentration | g/L | <0.8 | Not detectable |
| Colour | Pt—Co | 300-500 | 50-100 |

### EXAMPLE 8

### Removal of Anionic Impurities with Weak Anion Exchange Resin

A total of 84.29 L of demineralized extracted lactic acid solution was treated with a macroporous weak anion exchange resin column of 1 m length, 1.5″ diameter and 0.6 kg resin.

| | Unit | Before treatment | After treatment |
|---|---|---|---|
| Volume | L | 84.29 | 86.06 |
| pH | | <2 | <2 |
| Lactate concentration | g/L | 113-118 | 109-114 |
| Sulfate concentration | g/L | <0.8 | Not detectable |
| Ammonium concentration | g/L | Not detectable | Not detectable |

### EXAMPLE 9

### Nano-Filtration Membrane

An extracted lactic acid solution obtained from apparatus 62 (RO membrane) was subjected to two different processing path: i) direct concentration to 88±5% via apparatus 75, ii) treatment with apparatus 72 (Evaporator) follows by concentrated the permeate from apparatus 72 (NF membrane) with apparatus 75 (Evaporator).

| | Without NF treatment | With NF Treatment |
|---|---|---|
| Lactic Acid Concentration | 90.44% | 87.1% |
| Colour | 250 alpha | 75 alpha |

### EXAMPLE 10

### Product Concentration

88 L of diluted purified post polished lactic acid solution was concentrated to 88% lactic acid concentration.

| | Unit | Final Product |
|---|---|---|
| pH | | <1 |
| Lactate concentration | % | 85-90 |
| Sulfate concentration | ppm | <10 |
| Ammonium concentration | ppm | <10 |
| Colour | Pt—Co | 0 |
| Glucose concentration | ppm | Not Detectable |

Effect of Stabilizer in SLM

The stability of liquid membrane is highly related to the water transport across the membrane. Higher water transport would result in lower stability. Under experimental conditions, water transfers from receiving solution to source solution generally. Two new liquid membrane modules constructed with same batch of base polymer fibers were used. The organic layers impregnated in the micropores of the fibers have similar compositions except that one with addition of 0.001-0.02% non-ionic surfactant. The same source of L-lactic acid fermentation broth solution was used for the experiments.

| | Unit | Without Stabilizer | With Stabilizer |
|---|---|---|---|
| Lactate extraction | g/m2 · h | 29 | 24 |
| flux | | | |
| Extraction time | h | 20 | 20 |
| Initial source | L | 4 | 4 |
| solution volume | | | |
| Final source | L | 4.2 | 3.9 |
| solution volume | | | |
| Total sampling | L | ~0.1 | ~0.1 |
| volume | | | |
| Source solution | L | +0.3 | 0 |
| volume change | | | |
| Number of days | | 7 | More than 180 days |
| stable | | | |

Effect of Hydrogen Peroxide

The extracted solution obtained from apparatus 72 (NF membrane) was subjected to i) Hydrogen peroxide treatment; ii) without Hydrogen Peroxide treatment, follows by concentrating to 88±3% wt/wt Lactic Acid concentration

| | Without Hydrogen | With Hydrogen |
|---|---|---|
| | Peroxide treatment | Peroxide Treatment |
| Lactic Acid Concentration | 87.1% | 88.6% |
| Colour | 75 alpha | 25 alpha |

Heat Stability Test.

Concentrated 88% post polished lactic acid solution obtained (45 MT) from one of the embodiment of the above mention processes is subjected to heat stability test at 195±5° C. for different duration. The colour of the end solution was measured.

| Heating duration (min) | Colour (alpha) |
|---|---|
| Initial Colour | 5 |
| 15 | 50 |
| 30 | 70 |
| 60 | 75 |
| 90 | 95 |
| 120 | 100 |

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A process for recovery and purification of an organic acid from a fermentation broth containing a salt form of the organic acid, comprising in combination the steps of:
a. subjecting the fermentation broth to one of ultrafiltration and microfiltration to form a first permeate;
b. concentrating the first permeate to form a concentrated broth;
c. subjecting the concentrated broth to a supported liquid membrane for extraction of lactic acid into a separate stream comprising an extracted solution;
d. subjecting the extracted solution to activated carbon for colour removal, a cation exchange resin for demineralization, and an anion exchange resin for removal of anionic impurities to form a post polished organic acid;
e. filtering the post polished organic acid to remove impurities above a predetermined threshold; and
f. concentrating the post polished organic acid to a desired concentration.

2. The process of claim 1, wherein the supported liquid membrane comprises a base membrane and an organic layer impregnated on pores of the base membrane.

3. The process of claim 2, wherein the organic layer comprises a carrier, a co-extractant, a diluent and a stabilizer.

4. The process of claim 3, wherein the stabilizer is a form of ethoxylated fluorocarbon based surface-active agent that is non-ionic.

5. The process of claim 3, wherein the carrier comprises one of a primary amine, a secondary amine, a tertiary amine and an aromatic amine.

6. The process of claim 3, wherein the co-extractant is an aliphatic alcohol.

7. The process of claim 3, wherein the diluent comprises one of a hydrocarbon, a ketone, an ether and an ester.

8. The process of claim 5, wherein the amine has one or more side chains of branched, linear and cyclic C₄ to C₂₄.

9. The process of claim 6, wherein the aliphatic alcohol comprises one of linear and branched C₂ to C₂₉.

10. The process of claim 2, wherein the base membrane comprises one of polypropylene, polyethylene, polyvinyldifluoride, polyether sulfone and polysulfone.

11. The process of claim 1, wherein the supported liquid membrane has a hollow fiber configuration defining two sides, wherein one side is of an organic phase and the other side is of an aqueous phase.

12. The process of claim 11, wherein the organic phase contains more than two components.

13. The process of claim 1, wherein the ultrafiltration membrane has pores sizes within a range of from 0.1-0.01 μm and the microfiltration membrane has pores sizes within the range from 0.04-1 μm.

14. The process of claim 1, further comprising the step of feeding into the supported liquid membrane of step (c) one of water and water mixed with solutes.

15. The process of claim 1 wherein the step of subjecting the extracted solution to the cation exchange resin occurs prior to the step of subjecting the extracted organic acid solution to the anion exchange resin.

16. The process of claim 1 further comprising the steps of acidifying of the concentrated broth to a pH of 1 to 4.8, and separating salts from the concentrated broth prior to the step of subjecting the concentrated broth to the supported liquid membrane.

17. The process of claim 16 further comprising the step of separating precipitants by filtration prior to the step of subjecting the concentrated broth to the supported liquid membrane.

18. The process of claim 1 further comprising treating the post polished organic acid with an oxidizing agent to produce a heat stable organic acid.

19. The process of claim 1 wherein the organic acid is lactic acid.

20. The process of claim 1 further comprising the step of concentrating the extracted solution after the step of subjecting the concentrated broth to a supported liquid membrane and before the step of filtering the post polished organic acid to remove impurities above the predetermined threshold.

21. The process of claim 20 wherein the step of concentration the extracted solution is accomplished by use of a reverse osmosis membrane which permits only water of the extracted solution to flow through the membrane.

22. The process of claim 20 further comprising the step of concentrating the extracted solution after the step of subjecting the extracted solution to anion exchange and before the step of filtering the post polished organic acid to remove impurities above the predetermined threshold.

23. The process of claim 1 wherein the predetermined threshold of step (e) is greater than 100 Daltons.
